(19) United States
(12) Patent Application Publication
(10) Pub. No.: US 2019/0330380 A1
(43) Pub. Date: Oct. 31, 2019

(54) SYSTEMS AND METHODS FOR SEPARATING MIXTURES COMPRISING FLUOROCARBOXYLIC AND CARBOXYLIC ACIDS

(71) Applicant: Eastman Chemical Company, Kingsport, TN (US)

(72) Inventors: Robert Lin, Kingsport, TN (US); Glenn Lewis Shoaf, Jonesborough, TN (US); Venkateswarlu Bhamidi, Kingsport, TN (US); Shane Kipley Kirk, Church Hill, TN (US); Marty L. Neal, Blountville, TN (US); Steven J. Adams, Gray, TN (US)

(73) Assignee: Eastman Chemical Company, Kingsport, TN (US)

(21) Appl. No.: 16/465,256

(22) PCT Filed: Dec. 8, 2016
(86) PCT No.: PCT/US2016/065501

§ 371 Date: May 30, 2019

(51) Int. Cl. C08B 3/22; C07C 303/44; C07C 51/64; C07C 51/46; C08B 3/06; B01D 3/14; B01D 9/00

(52) U.S. Cl. CPC C08B 3/22; C07C 303/44; C07C 51/64; C07C 51/46; B01D 2009/0086; B01D 3/143; B01D 9/0059; C07B 2200/13; C08B 3/06

(57) ABSTRACT

Processes and systems for separating a mixture including at least one fluorocarboxylic acid and at least one carboxylic acid are provided herein. In some aspects, the present invention relates to processes and systems configured to provide a purified stream of fluorocarboxylic acid and a purified stream of carboxylic acid, even when the mixture includes an azeotrope, a pinch point, and/or a eutectic mixture of the fluorocarboxylic acid and carboxylic acid. Systems as described herein may include, for example, at least one distillation zone and at least one fractional crystallization zone arranged in series and configured to provide highly purified product streams having compositions heretofore unachievable by conventional means.

### BACKGROUND

### 1. Field of the Invention

The present invention relates generally to processes and systems for the separation and purifications of mixtures including at least one fluorocarboxylic acid and at least one carboxylic acid.

### 2. Description of Related Art

Fluorocarboxylic acids are useful in many processes in the chemical industry. For example, fluorocarboxylic acids and anhydrides are used extensively as a preparative agent in general organic chemistry, such as, for example, in carbohydrate chemistry and in various types of esterification reactions. For example, trifluoroacetic anhydride and trifluoroacetic acid have been successfully employed as catalysts in the production of cellulose esters. For example, as described in PCT Application Publication No. WO91/14709 to Buchanan et al., a variety of cellulose esters, including cellulose acetates, cellulose propionates, cellulose butyrates, cellulose hexanoates, and cellulose benzoates, can be prepared by combining trifluoroacetic anhydride with cellulose and a corresponding carboxylic acid and/or anhydride to form an esterified product. In addition to the product cellulose ester, the resulting reaction mixture typically includes a residual mixture of trifluoroacetic acid and carboxylic acid.

Desirably, the trifluoroacetic acid recovered from the reaction mixture could be converted to trifluoroacetic anhydride and recycled back to the process. However, trifluoroacetic acid and acetic acid form both an azeotropic mixture and a eutectic mixture, which makes separating the stream into its constituent components impossible with conventional separation systems. As a result, many processes tend to utilize trifluoroacetic acid on a “once through” basis and simply dispose of the mixture of trifluoroacetic acid and carboxylic acid once the product cellulose ester has been recovered. No process has successfully established a workable, efficient system for the recovery of trifluoroacetic acid from a mixture of carboxylic acid to form purified product streams of each.

Thus, a need exists for an efficient method for separating mixtures including fluorocarboxylic acids and carboxylic acids into purified product streams, even when such mixtures form azeotropes, pinch points, and/or eutectic mixtures. Ideally, such methods could be performed in pilot-scale and commercial-scale facilities and using existing equipment. Further, it would be advantageous if such methods could be easily implemented in various processes for synthesizing organic materials, including cellulose esters, in order to maximize production, while minimizing cost.

### SUMMARY

One embodiment of the present invention concerns a method for separating a fluorocarboxylic acid and a carboxylic acid. The method comprises the steps of separating a feed stream comprising the fluorocarboxylic acid and the carboxylic acid in a first separation zone to provide at least a first pure component stream and a first mixed component stream. The first separation zone comprises a distillation zone or a crystallization zone. The method also comprises separating at least a portion of the first mixed component stream in a second separation zone to provide at least a second pure component stream and a second mixed component stream, wherein the second separation zone comprises the other of a distillation zone and a crystallization zone, and recycling at least a portion of the second mixed component stream from the second separation zone to the first separation zone.

Another embodiment of the present invention concerns a method for separating a fluorocarboxylic acid and a carboxylic acid. The method comprises introducing a first fluid stream comprising at least one fluorocarboxylic acid and at least one carboxylic acid into a distillation zone and separating the first fluid stream in the distillation zone to provide an overhead stream and a bottoms stream. The method comprises introducing at least a portion of the bottoms stream into a crystallization zone, separating at least a portion of the bottoms stream in the crystallization zone to form a predominantly solid phase and a predominantly liquid phase, and introducing at least a portion of the predominantly liquid phase into the distillation zone.

Yet another embodiment of the present invention concerns a system for separating a fluorocarboxylic acid and a carboxylic acid. The system comprises a distillation zone for receiving a first fluid stream comprising at least one fluorocarboxylic acid and at least one carboxylic acid. The distillation zone is configured to separate the first fluid stream into a predominantly vapor first pure component overhead stream and a predominantly liquid first mixed component bottoms stream. The system comprises a crystallization zone for receiving the first mixed component bottoms stream. The crystallization zone is configured to separate the first mixed component bottoms stream into a predominantly solid pure component stream and a predominantly liquid mixed component stream. The system comprises a recycle conduit for passing at least a portion of the predominantly liquid mixed component stream from the crystallization zone to the distillation zone.

### BRIEF DESCRIPTION OF THE DRAWINGS

Various embodiments of the present invention are described in detail below with reference to the attached drawing Figures, wherein:

FIG. 1 is a schematic diagram of the major stages of a separation system configured according to one or more embodiments of the present invention;

FIG. 2 is a graphical depiction of the vapor-liquid and solid-liquid equilibrium data for a binary system including trifluoroacetic acid and acetic acid;

FIG. 3 is a schematic diagram of a separation system configured according to one or more embodiments of the present invention, particularly illustrating embodiments wherein the first separation zone includes a distillation zone;

FIG. 4 is a schematic diagram of a separation system configured according to one or more embodiments of the present invention, particularly illustrating embodiments wherein the first separation zone includes a crystallization zone; and

FIG. 5 is a schematic diagram of a separation system simulated in the process model described in the Example.

### DETAILED DESCRIPTION

Various embodiments of the present invention relate to methods and systems for separating and purifying mixtures comprising at least one fluorocarboxylic acid and at least one carboxylic acid. Systems and methods as described herein may be particularly useful when the acid components present in the mixture form an azeotrope and/or eutectic point with one another. Conventional separation systems are often ineffective at purifying such mixtures because, for example, azeotropes and eutectic points represent physical compositional “boundaries,” that cannot be crossed using traditional separation techniques, such as simple distillation. Systems and methods as described herein are capable of overcoming the purification limitations caused by the presence of azeotropes and eutectic points to provide separate, highly purified product streams.

Mixtures separable by embodiments of the present invention can comprise at least one fluorocarboxylic acid and at least one carboxylic acid that exhibit one or more of an azeotrope, a pinch point, and a eutectic point. As used herein, the term “azeotrope” refers to the constant-boiling composition of a vapor-liquid mixture of two or more components that cannot be separated by simple distillation. At an azeotrope, the relative volatility of the mole fraction of one component is equal to the relative volatility of the other. As a result, increasing the temperature of such mixtures does not change the composition of the vapor or liquid phases. This limits the ability of a stream having such a composition to be separated into its individual components, thereby creating a compositional “boundary,” which cannot be crossed by simple distillation techniques.

As used herein, the term “pinch point” refers to a composition at which the relative volatilities of two components are not equal, but are close enough that the driving force for mass transfer is minimized so that separation of the mixture by simple distillation is not practical. For example, a pinch point exists when the relative volatilities of the components are within about 0.05, within about 0.025, or within about 0.01, of one another. As a result, separation of such a stream by simple distillation, although theoretically possible, requires a significant number of theoretical stages and an excessive amount of energy and expense to perform, thereby making it highly impractical and undesirable.

As used herein, the term “eutectic point” refers to the solid-liquid composition of a mixture of two or more components that has the lowest possible complete melting temperature. At the eutectic point, further lowering the temperature of the eutectic mixture does not change the composition of the solid or liquid phase. As a result, a mixture having a composition at the eutectic point may not be further purified by conventional crystallization.

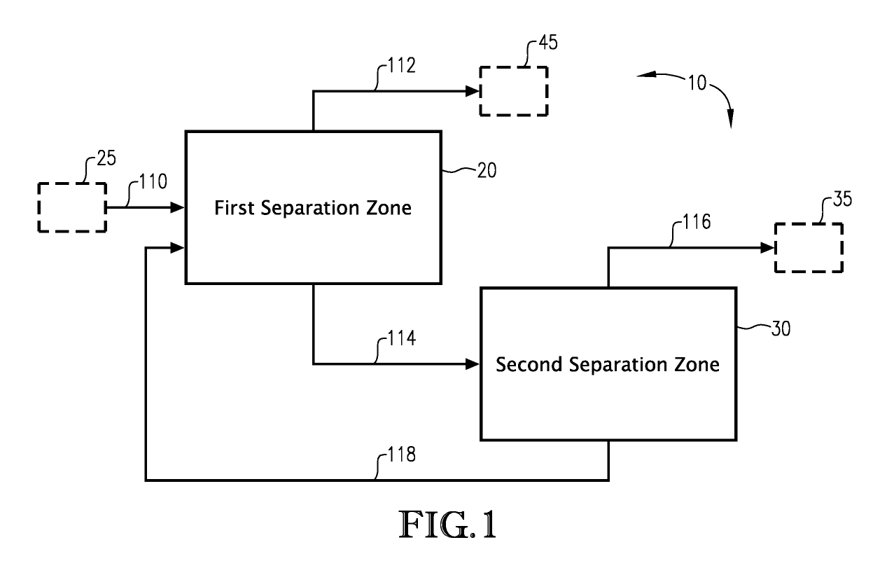

Turning initially to FIG. 1, a schematic overview of the main steps of a separation system 10 configured according to one or more embodiments of the present invention is provided. Separation system 10, which may be used to separate a mixture of a fluorocarboxylic acid and a carboxylic acid, includes a first separation zone 20 and a second separation zone 30, as well as optional pre-treatment and post-treatment zones 25 and 35. Each of first and second separation zones 20, 30 are configured to separate a feed stream into at least one pure component stream and at least one mixed component stream. In combination, separation zones 20, 30 may be configured to “break” a compositional boundary imposed by an azeotrope, pinch point, and/or eutectic point in order to provide highly purified product streams.

In some embodiments, separation system 10 shown in FIG. 1 may be particularly useful for separating a feed stream comprising a mixture of at least one fluorocarboxylic acid and at least one carboxylic acid. As used herein, the term “fluorocarboxylic acid,” generally refers to an organic compound including at least one carbon atom, at least one fluorine atom, and at least one hydroxyl group (—OH). The fluorocarboxylic acid or acids present in the feed stream in line 110 may include from 1 to 10, from 1 to 8, or from 1 to 6 total carbon atoms, and may comprise a linear or branched aliphatic chain.

In some embodiments, one or more fluorocarboxylic acids present in the feed stream can have the following general formula (I):

CₙFₘHₚ—COOH (I),

wherein n is an integer in the range of from 1 to 9, from 1 to 8, or from 1 to 6, m is an integer in the range of from 1 to 2n+1, and p is an integer in the range of from 0 to 2n, and wherein p+m=2n+1. In some embodiments, the fluorocarboxylic acid be a perfluorocarboxylic acid, defined by formula (I) above, when p=0 and m=2n+1.

In other embodiments, one or more fluorocarboxylic acids may comprise a fluorinated sulfonic acid of the general formula (II):

R₁—SO₃H (II),

wherein R₁ is a carbon group including at least one fluorine atom. In some embodiments, R₁ can include at least two, or three fluorine atoms and may comprise in the range of from 1 to 9, from 1 to 8, or from 1 to 6 carbon atoms.

Additionally, in some embodiments, the fluorocarboxylic acid may comprise a fluorinated carboxylic acid in which all of the fluorine atoms are present on the carbon atom relative to the carboxyl group (—COOH), as described by general formula (III):

F₃C—(CH₂)ₙ₋₁—COOH (III),

wherein n is an integer in the range of from 1 to 9, from 1 to 8, or from 1 to 6.

Examples of suitable fluorocarboxylic acids can include, but are not limited to, difluoroacetic acid (DFA), chlorodifluoroacetic acid (CDFA), trifluoroacetic acid (TFA), 3,3,3-trifluoropriopionic acid, pentafluoropropionic acid, heptafluorobutyric acid, perfluoropentanoic acid, perfluorohexanoic acid, perfluoroheptanoic acid, perfluorooctanoic acid, and combinations thereof. When the fluorocarboxylic acid is a fluorinated sulfonic acid, it may also include trifluoromethanesulfonic, or triflic, acid. In some embodiments, the fluorocarboxylic acid may include halogen atoms other than fluorine, including, for example, chlorine or bromine. The fluorocarboxylic acid may originate from any suitable source and may, in some embodiments, be derived from the hydrolysis of its corresponding acid anhydride.

In certain embodiments, the feed stream in line 110 may comprise at least about 0.5, at least about 1, at least about 5, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, or at least about 95 weight percent of one or more fluorocarboxylic acids, based on the total weight of the feed stream in line 110. In addition, or alternatively, the feed stream in line 110 may comprise not more than about 99, not more than about 95, not more than about 90, not more than about 85, not more than about 80, not more than about 75, not more than about 70, not more than about 65, not more than about 60, not more than about 55, not more than about 50, not more than about 45, not more than about 40, not more than about 35, not more than about 30, not more than about 25, not more than about 20, not more than about 15, not more than about 10, not more than about 5, or not more than about 1 weight percent of one or more fluorocarboxylic acids, based on the total weight of the feed stream in line 110.

As used herein, the term “carboxylic acid,” refers to an organic acid compound defined by the following formula (III):

R₂—COOH (IV),

wherein R₂ is hydrogen or a carbon group including from 1 to 20, from 1 to 18, from 1 to 16 carbon atoms. The R₂ group may include a linear or branched aliphatic chain, or it may include one or more aromatic groups. Examples of suitable carboxylic acids can include, but are not limited to, acetic acid, i-propionic acid, n-propionic acid, n-butyric acid, i-butyric acid, trimethylacetic acid, valeric acid, hexanoic acid, nonanoic acid, benzoic acid, napthanonic acid, and combinations thereof. As used herein, the term “carboxylic acid” does not encompass compounds that include a fluorine atoms, but carboxylic acids as described herein can include atoms of another halogen such as, for example, chlorine or bromine. The carboxylic acid may originate from any suitable source and may, in some embodiments, be derived from the hydrolysis of its corresponding acid anhydride.

The feed stream in line 110 may include at least one carboxylic acid. For example, the feed stream in line 110 may comprise at least about 0.5, at least about 1, at least about 5, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, or at least about 95 weight percent of one or more carboxylic acids, based on the total weight of the feed stream in line 110. In addition, or alternatively, the feed stream in line 110 may comprise not more than about 99, not more than about 95, not more than about 90, not more than about 85, not more than about 80, not more than about 75, not more than about 70, not more than about 65, not more than about 60, not more than about 55, not more than about 50, not more than about 45, not more than about 40, not more than about 35, not more than about 30, not more than about 25, not more than about 20, not more than about 15, not more than about 10, not more than about 5, or not more than about 1 weight percent of one or more carboxylic acids, based on the total weight of the feed stream in line 110. Although described herein as including a single fluorocarboxylic acid and single carboxylic acid, it should be understood that feed streams separable by systems and methods as described herein may also include at least one, at least two, or at least three additional fluorocarboxylic acids and/or carboxylic acids, as long as the presence of such materials does not adversely impact the separation of the final fluorocarboxylic acid and carboxylic acid streams.

In some embodiments, each individual carboxylic acid and fluorocarboxylic acid may be present in the feed stream in line 110 in an amount of at least about 5, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90 weight percent, based on the combined weight of all carboxylic and fluorocarboxylic acids present in the feed stream. Additionally, or in the alternative, each individual carboxylic acid and fluorocarboxylic acid may be present in the mixture in an amount of not more than about 90, not more than about 85, not more than about 80, not more than about 75, not more than about 70, not more than about 65, not more than about 60, not more than about 55, not more than about 50, not more than about 45, not more than about 40, not more than about 35, not more than about 30, not more than about 25, not more than about 20, not more than about 15, or not more than about 10 weight percent, based on the combined weight of all carboxylic and fluorocarboxylic acids present in the feed stream.

Optionally, the feed stream in line 110 may include one or more components other than the fluorocarboxylic acid and the carboxylic acid. When present, the total amount of these additional components may be at least about 0.5, at least about 1, at least about 2, or at least about 5 weight percent and/or not more than about 20, not more than about 15, not more than about 10, or not more than about 8 weight percent, based on the total weight of the feed stream. In some embodiments, the total amount of components other than the fluorocarboxylic acid and carboxylic acid can be not more than about 5, not more than about 3, not more than about 2, not more than about 1, or not more than about 0.5 weight percent, based on the total weight of the mixture. In some embodiments, the mixture may be a binary mixture of the fluorocarboxylic acid and carboxylic acid and may include not more than about 0.5, not more than about 0.25, not more than about 0.10, or not more than about 0.05 weight percent of components other than the fluorocarboxylic acid and carboxylic acid, based on the total weight of the mixture.

Examples of components other than the fluorocarboxylic acid and carboxylic acid that may be present in the feed stream in line 110 can include, but are not limited to, water, anhydrides of one or both of the fluorocarboxylic acid and carboxylic acid, and combinations thereof. Other types of additional components can include various types of cellulose esters including, but not limited to, cellulose acetates, cellulose propionates, cellulose butyrates, cellulose hexanoates, and cellulose benzoates. Any number or type of components other than the fluorocarboxylic acid and carboxylic acid may be present in feed stream 110, as long as the additional components do not prevent or adversely impact the separation of the fluorocarboxylic acid and carboxylic acid within separation system 10. Optionally, the amounts of one or more of these or other components may be reduced using any suitable process in pre-treatment zone 25.

In some embodiments, the combined amount of fluorocarboxylic acid and carboxylic acid present in the feed stream in line 110 may be at least about 5, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, or at least about 95 weight percent, based on the total weight of the feed stream. Additionally, or in the alternative, the total amount of carboxylic and fluorocarboxylic acids present in the feed stream in line 110 may be not more than about 99, not more than about 95, not more than about 90, not more than about 85, not more than about 80, not more than about 75, not more than about 70, not more than about 65, not more than about 60, not more than about 55, not more than about 50, not more than about 45, not more than about 40, not more than about 35, not more than about 30, not more than about 25, or not more than about 20 weight percent, based on the total weight of the feed stream.

Referring again to FIG. 1, the feed stream in line 110 may be introduced into first separation zone 20, wherein it may be separated to form a first pure component stream in line 112 and a first mixed component stream in line 114. The first pure component stream in line 112 may be enriched in one of the fluorocarboxylic acid and carboxylic acid present in the feed stream in line 110, and the first mixed component stream in line 114 may include a mixture of the fluorocarboxylic acid and carboxylic acid. As used herein, the term “enriched,” as it applies to a process stream removed from a zone, column, or other vessel, refers to the process stream that has a higher amount (by weight) of a given component than the amount (by weight) of that component present in each of the other individual stream or streams removed from the same zone, column, or vessel. For example, an overhead vapor stream withdrawn from a distillation column may be “enriched” in component A if the overhead vapor stream includes a total weight of component A that is higher than the total weight of component A present in each of the bottoms liquid stream and any side streams also withdrawn from the column, on an individual basis. For example, if a feed stream including 7 pounds per hour (lb/h) of component A were divided into an overhead stream including 5 lb/h of component A and a bottoms stream including 2 pounds per hour (lb/h) of component A, the overhead stream would be said to be enriched in component A.

In some embodiments, the pure component stream in line 112 may include at least about 55, at least about 60, at least about 65, at least about 70, at least about 75, at least about 80, at least about 85, at least about 90, at least about 92, at least about 95, or at least about 97 weight percent of the fluorocarboxylic acid or the carboxylic acid, based on the total weight of the pure component stream. This may represent an amount of fluorocarboxylic acid or carboxylic acid that is at least about 40, at least about 50, at least about 60, at least about 70, at least about 80, or at least about 90 percent of the total amount, by weight, of the fluorocarboxylic acid or carboxylic acid introduced into first separation zone 20 via line 110.

The mixed component stream in line 114 may comprise a mixture of the fluorocarboxylic acid and carboxylic acid. In some embodiments, each of the fluorocarboxylic acid and the carboxylic acid may be present in the mixed component stream in line 114 can be at least about 5, at least about 10, at least about 20, at least about 25, at least about 30, or at least about 35 weight percent and/or not more than about 60, not more than about 55, not more than about 50, not more than about 45, not more than about 40, or not more than about 35 percent, based on the total weight of the mixed component stream. The ratio of the fluorocarboxylic acid to carboxylic acid in the mixed component stream in line 114 may be at least about 0.5:1, at least about 0.75:1, at least about 1.1:1, or at least about 1.5:1 and/or not more than about 3:1, not more than about 2.5:1, not more than about 2:1, or not more than about 0.95:1. In some embodiments, the composition of the mixed component stream in line 114 may approach an azeotrope, a pinch point, or a eutectic composition of the fluorocarboxylic acid and carboxylic acid present in the feed stream in line 110.

As used herein, the terms “approaching” or “approach” used in reference to a composition means within about 15 percent of a certain composition. Therefore, a stream having a composition “approaching” an azeotrope has a composition within about 15 percent of the azeotropic composition. For example, if the azeotropic composition for a given stream includes 80 weight percent of component A, a stream having a composition approaching the azeotrope would comprise at least 65 percent of component A or not more than 95 percent of component A. The term “within” encompasses values both higher and lower by a given amount. In some embodiments, a stream having a composition approaching the azeotrope can have a composition within about 10 or within about 5 percent of the azeotropic composition. In some embodiments, the stream may have a composition at an azeotrope, eutectic point, or pinch point.

Referring again to FIG. 1, the mixed component stream in line 114 withdrawn from first separation zone 20 may be introduced into second separation zone 30, wherein it may be separated into another pure component stream in line 116 and another mixed component stream in line 118. The pure component stream in line 116 may be enriched in the other of the fluorocarboxylic acid and carboxylic acid. For example, in some embodiments, the pure component stream in line 116 may include at least about 55, at least about 60, at least about 65, at least about 70, at least about 75, at least about 80, at least about 85, at least about 90, at least about 92, at least about 95, or at least about 97 weight percent of the carboxylic or fluorocarboxylic acid, based on the total weight of the pure component stream. This may represent at least about 40, at least about 50, at least about 60, at least about 70, at least about 80, or at least about 90 percent of the total amount, by weight, of the carboxylic acid or fluorocarboxylic acid introduced into second separation zone 30 via line 114, and at least about 40, at least about 50, at least about 60, at least about 70, at least about 80, or at least about 90 percent of the total amount, by weight, of the carboxylic acid or fluorocarboxylic acid introduced into first separation zone 20 via line 110.

The mixed component stream withdrawn from second separation zone 30 in line 118 may comprise another mixture of the fluorocarboxylic acid and carboxylic acid. In some embodiments, the mixed component stream in line 118 may include at least about 5, at least about 10, at least about 20, at least about 25, or at least about 30 weight percent and/or not more than about 60, not more than about 55, not more than about 50, not more than about 45, not more than about 40, or not more than about 35 weight percent of each of the fluorocarboxylic acid and carboxylic acid, based on the total weight of the mixed component stream. The ratio of the fluorocarboxylic acid to carboxylic acid in the mixed component stream in line 118 may be at least about 0.5:1, at least about 0.75:1, at least about 1.1:1, or at least about 1.5:1 and/or not more than about 3:1, not more than about 2.5:1, not more than about 2:1, or not more than about 0.95:1. In some embodiments, the composition of the mixed component stream in line 118 may approach the composition of an azeotrope, a pinch point, or a eutectic point for the fluorocarboxylic acid and carboxylic acid present in the feed stream in line 110.

The composition of the mixed component stream in line 118 may have a different composition than that of the mixed component stream in line 114. Additionally, the pure component streams in lines 112 and 116 may be enriched in different components. For example, in some embodiments, the pure component stream in line 112 may be enriched in the carboxylic acid, while the pure component stream in line 114 may be enriched in the fluorocarboxylic acid. Alternatively, the first mixed component stream in line 114 may be enriched in the fluorocarboxylic acid, while the second mixed component stream in line 118 may be enriched in the carboxylic acid. When the composition of the first mixed component stream in line 114 is approaching an azeotrope or a pinch point, the composition of the second mixed component stream in line 118 may approach a eutectic point.

Alternatively, in when the composition of the first mixed component stream in line 114 is approaching a eutectic point, the composition of the stream in line 118 may approach an azeotrope or a pinch point.

According to embodiments of the present invention, at least one of first and second separation zones 20 and 30 may be configured to separate a feed stream using vapor-liquid separation, while the other of first and second separation zones 20 and 30 may be configured to separate a feed stream using solid-liquid separation. For example, first separation zone 20 can be a distillation zone and second separation zone 30 can be a crystallization zone. Alternatively, first separation zone 20 can be a crystallization zone and second separation zone 30 can be a crystallization zone. Several embodiments of separation systems 10 including first and second separation zones 20 and 30 will be discussed in detail shortly.

As shown in FIG. 1, separation system 10 may further comprise an optional pre-treatment zone 25 for performing one or more additional processing steps on the feed in line 110 prior to its introduction into first separation zone 20. In the same or other embodiments, the separation system 10 shown in FIG. 1 may also include one or more optional post-treatment zones 35, 45 for performing one or more additional processing steps on the pure component streams in lines 112 and 116. Examples of suitable processing steps may include, but are not limited to, vapor-liquid separation, filtration, centrifugation, reverse osmosis, perevaporation, membrane separation, ion exchange, dehydration, heating, cooling, and combinations thereof.

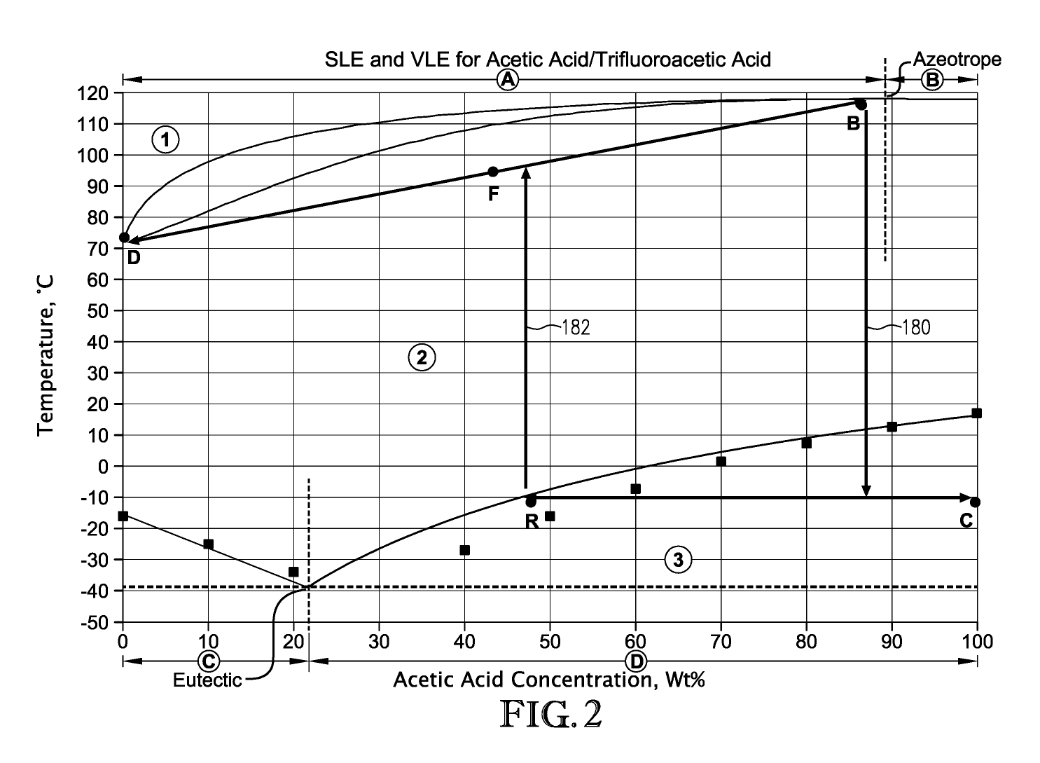

Turning now to FIG. 2, a phase diagram including the vapor-liquid equilibrium data and solid-liquid equilibrium data for a binary system of acetic acid and trifluoroacetic acid is provided. Although shown as a function of acetic acid concentration, it should be understood that such data could also be provided as a function of trifluoroacetic acid concentration. As discussed above, other components may be present in the feed stream being separated, as long as such components do not adversely interfere with the phase behavior shown in FIG. 2. It should be further understood that, although described with respect to one embodiment of a mixture including trifluoroacetic acid and acetic acid, similar analyses may be performed using equilibrium data for any system including a fluorocarboxylic and a carboxylic acid according to embodiments of the present invention.

As shown in FIG. 2, acetic acid and trifluoroacetic acid exhibit an azeotrope at a temperature of approximately 118° C., at which point the mass fraction of acetic acid in the vapor and liquid phases is approximating 0.87, or 87 weight percent. Additionally, acetic acid and trifluoroacetic acid also exhibit a eutectic point at a temperature of approximately −40° C. At the eutectic point, the mass fraction of acetic acid in the solid and liquid phases is approximately 0.28, or 28 weight percent. For a given feed composition, the phase equilibrium data shown in FIG. 2 may be used to predict the compositions of the streams in lines 112, 114, 116, and 118 when the feed stream is subjected to distillation or crystallization in first and second separation zones 20 and 30 shown in FIG. 1.

For example, as discussed previously, one of first separation zone 20 and second separation zone 30 can comprise a distillation zone. In some embodiments, the mass fraction (or weight percent) of acetic acid in the feed stream, based on the total weight of acetic acid and trifluoroacetic acid, introduced into a distillation zone may be lower than the mass fraction (or weight percent) of acetic acid at the azeotrope, as shown by Region A in FIG. 2. In such embodiments, the pure component stream withdrawn from the distillation zone will be enriched in trifluoroacetic acid and the mixed component stream will have a composition approaching the azeotrope. Conversely, when the mass fraction (or weight percent) of acetic acid in the feed stream, based on the total weight of acetic acid and trifluoroacetic acid, introduced into a distillation zone is higher than the mass fraction (or weight percent) of acetic acid at the azeotrope, as shown by Region B in FIG. 2, the pure component stream withdrawn from the separation zone may be enriched in acetic acid and the mixed component stream may have a composition approaching the azeotrope. Such results may occur whether first or second separation zone 20, 30 includes a distillation zone.

Further, one of first and second separation zones 20 and 30 can comprise a crystallization zone. When the feed stream is introduced into a crystallization zone and the mass fraction of acetic acid in the feed stream is lower than the mass fraction of acetic acid at the eutectic point, which is shown by Region C in FIG. 2, the pure component stream will be enriched in trifluoroacetic acid, and the mixed component stream will have a composition approaching the eutectic point. Conversely, when the mass fraction of acetic acid in the feed stream introduced a crystallization zone is higher than the mass fraction of acetic acid at the eutectic point, as shown by Region D in FIG. 2, the pure component stream will be enriched in acetic acid, and the mixed component stream may have a composition approaching the eutectic point composition. Such results may occur whether first or second separation zone 20, 30 includes a crystallization zone.

As shown in FIG. 2, whether the mass fraction of acetic acid is higher or lower than at the azeotrope or higher or lower than the at the eutectic point, no single distillation or crystallization zone will provide highly purified streams of acetic and trifluoroacetic acid. For example, while a distillation zone may provide an overhead vapor stream of nearly purified acetic acid (when, for example, the mass fraction of acetic acid is higher than the mass fraction of acetic acid at the azeotrope) or nearly purified trifluoroacetic acid (when, for example, the mass fraction of acetic acid is less than the mass fraction of acetic acid at the azeotrope), the composition of the bottoms stream is limited by the azeotrope, which includes approximately 13 weight percent trifluoroacetic acid. Similarly, while a crystallization zone may provide a solid composition of nearly purified acetic acid (when, for example, the mass fraction of acetic acid is higher than the mass fraction of acetic acid at the eutectic point) or nearly purified trifluoroacetic acid (when, for example, the mass fraction of acetic acid is lower than the mass fraction of acetic acid at the eutectic point), the composition of the liquor phase is limited by the eutectic point and will not have less than 27 weight percent trifluoroacetic acid.

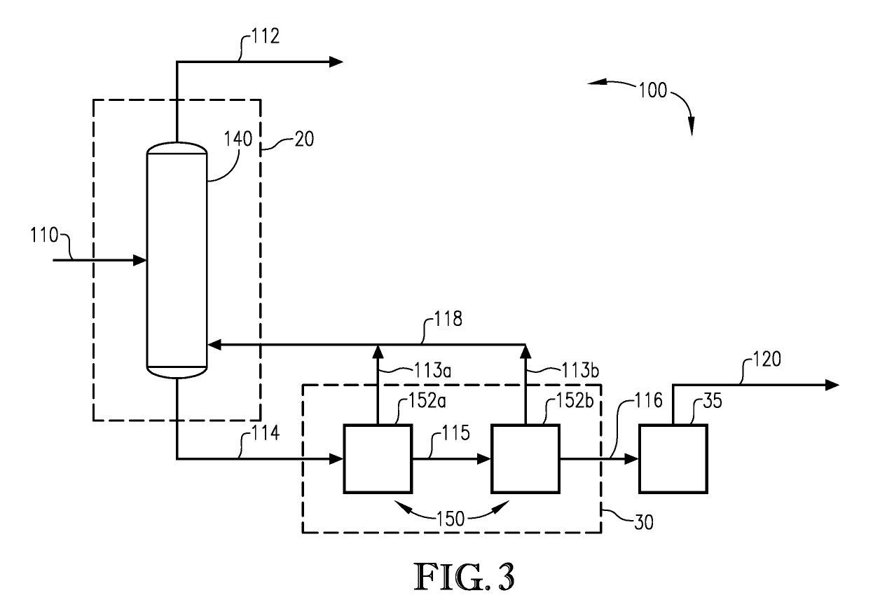

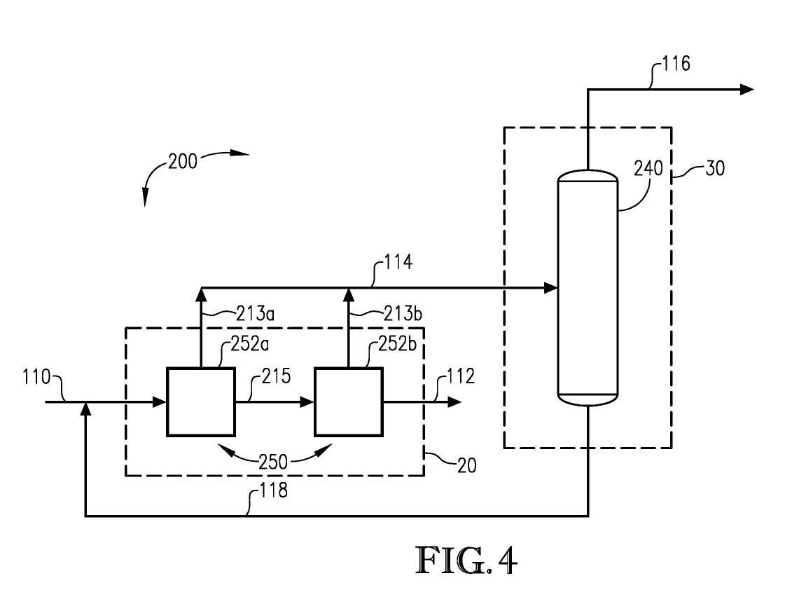

According to embodiments the present invention, the compositional boundaries imposed by azeotropes and/or eutectic points within mixtures of fluorocarboxylic and carboxylic acids may be circumvented, or “broken,” by using a combination of distillation and crystallization in order to achieve substantially purified streams of fluorocarboxylic acid and carboxylic acid. In the system 10 shown in FIG. 1, one of first separation zone 20 and second separation zone 30 is a distillation zone and the other of first separation zone 20 and second separation zone 30 is a crystallization zone. For example, in some embodiments, first separation zone 20 is a distillation zone and second separation zone 30 is a crystallization zone, while, in other embodiments, first separation zone 20 is a crystallization zone and second separation zone 30 is a distillation zone. Specific embodiments of such systems will now be described in detail below, with respect to FIGS. 3 and 4.

Turning initially to FIG. 3, one embodiment of a separation system 100 is shown wherein first separation zone 20 is a distillation zone and second separation zone 30 is a crystallization zone. As shown in FIG. 3, first separation zone 20 comprises at least one vapor-liquid separator 140 and second separation zone 30 comprises at least one crystallizer 150. The fluorocarboxylic acid and carboxylic acid present in the feed stream in line 110 may exhibit at least one azeotrope and at least one eutectic point, and the composition of the feed stream in line 110 may comprise the carboxylic acid in an amount higher or lower than the amount of carboxylic acid at the azeotrope. Additionally, the amount of fluorocarboxylic acid in the feed stream in line 110 may be lower or higher than at the azeotrope.

As shown in FIG. 3, a feed stream in line 110 is introduced into vapor-liquid separator 140 of first separation zone 20, wherein it may be separated to into a predominantly vapor overhead stream and a predominantly liquid bottoms stream. As used herein, the term “predominantly” means at least 55 percent, so that, for example, a predominantly liquid stream includes at least 55 weight percent liquid. The overhead vapor stream withdrawn from the vapor-liquid separator in line 112 may comprise a pure component stream enriched in one of the carboxylic acid and the fluorocarboxylic acid, while the bottoms liquid stream in line 114 may comprise a mixed component stream including fluorocarboxylic acid and carboxylic acid in amounts approaching the azeotrope composition. In some embodiments, the overhead vapor stream in line 112 can include at least about 75, at least about 80, at least about 85, at least about 90, or at least about 95 weight percent of one of the carboxylic acid and fluorocarboxylic acid, and not more than about 25, not more than about 20, not more than about 15, not more than about 10, or not more than about 5 weight percent of the other.

Although shown in FIG. 3 as including a single vapor-liquid separator 140, a distillation zone configured according to embodiments of the present invention may include at least 2, at least 3, or 4 or more vapor liquid separators arranged in series or in parallel. As used herein, the term “vapor-liquid separator” refers to a device configured to provide at least one fractional theoretical stage of separation. Examples of suitable vapor-liquid separators include, but are not limited to, distillation columns, flash pots, falling film evaporators, and combinations thereof. Each vapor-liquid separator may be operated in a batch, a semi-batch, a semi-continuous, or a continuous manner.

Additionally, vapor-liquid separator 140 shown in FIG. 3 may include any suitable type of internal contacting structure for facilitating mass and energy transfer between the vapor and liquid phases within the interior of the vessel. Examples of suitable internal contacting structures can include, but are not limited to, random packing, structured packing, vapor-liquid contacting trays, and combinations thereof. In some embodiments, one or more of the vapor-liquid separators may be substantially empty and may have no internals. In addition to vapor-liquid separator 140 shown in FIG. 3, first separation zone 20 may further include various auxiliary equipment such as heaters, condensers, piping, valves, and pumps needed for operation of the vapor-liquid separator. Examples of suitable heaters may include, but are not limited to, sand baths, oil baths, indirect heaters, and steam heaters, while suitable condensers may include water coolers or refrigerated coolers. Optionally, a portion of the warmed streams from one or more heaters may be returned to vapor-liquid separator 140 as a boil-up or stripping vapor, while a portion of the cooled streams from one or more condensers may be returned to vapor-liquid separator 140 as reflux (not shown in FIG. 3).

As shown in FIG. 3, the bottoms liquid stream in line 114 withdrawn from vapor-liquid separator 140 may be introduced into a crystallizer 150 of second separation zone 30, wherein the stream may be cooled and at least partially crystallized to form a solid phase and a liquor stream. Crystallizer 150 may utilize any suitable type of crystallization including, but not limited to, fractional crystallization, falling film crystallization, static crystallization, melt crystallization, suspension crystallization, and combinations thereof.

As shown in the embodiment depicted in FIG. 3, crystallizer 150 may be a multi-stage fractional crystallizer including at least a first and second crystallization stage 152a, 152b. Although shown as including two stages, it should be understood that crystallizers having more than two stages or a single stage could also be utilized according to embodiments of the present invention. For example, crystallizer 150 may include 1, at least 2, at least 3, at least 4, at least 5, or 6 or more crystallization stages. Crystallizer 150 may be operated in a counter-current or co-current manner and may be a continuous or discontinuous process. In some embodiments, crystallizer 150 may include a plurality of tubes, pipes, baffles, or plates that define a surface along which the liquid to be crystallized flows, while a heating or cooling medium flow along an adjacent surface. The heat transfer between the heat transfer medium and the liquid causes freezing of one of the components of the liquid stream, thereby producing a solid phase enriched in one component and a liquor phase enriched in the other. When the crystallizer is a multi-stage crystallizer, liquid product from one crystallizer or crystallization stage may be introduced as feed to an adjacent crystallizer or crystallization stage. One example of a crystallization process and device suitable for use in embodiments of the present invention is described in U.S. Reissue Pat. No. 32,241, the entirety of which is incorporated herein by reference to the extent not inconsistent with the present disclosure.

As shown in FIG. 3, the mixed component stream in line 114, which includes a mixture of fluorocarboxylic acid and carboxylic acid having a composition approaching the azeotropic composition, may be optionally cooled in a heat exchanger (not shown) before being introduced into the first stage 152a of crystallizer 150. In first stage 152, the stream in line 114 may be at least partially frozen to thereby provide a predominantly liquid phase in line 113a and a predominantly solid phase in line 115. The predominantly liquid phase in line 113a may be enriched in the fluorocarboxylic acid or the carboxylic acid, while the solids phase may comprise a mixture of fluorocarboxylic acid and carboxylic acid approaching the eutectic point composition.

As shown in FIG. 3, the solids phase in line 115 may then be introduced into a second stage 152b of crystallizer 150, wherein the stream may be subjected to further crystallization to provide a second liquid phase in line 113b and a second solids phase in line 116. The second liquid phase in line 113b, which may be enriched in the fluorocarboxylic acid or the carboxylic acid, may be combined with the first liquid phase in line 113a to form a combined liquid phase in line 118. This combined liquid phase in line 118 withdrawn from the crystallizer 150 may be optionally heated in a heat exchanger (not shown) and returned to vapor-liquid separator 140 as shown in FIG. 3. In some embodiments, the mixed component stream in line 118 may comprise fluorocarboxylic acid and carboxylic acid in amounts approaching the eutectic point composition.

The pure component solid phase in line 116 may optionally be subjected to further processing such as, for example, a melting step or a purification step, in a post-treatment zone 35 as shown in FIG. 3 to provide a final product stream in line 120. The final product stream in line 120, which can be a predominantly liquid stream, can include at least about 75, at least about 80, at least about 85, at least about 90, at least about 95 weight percent of the one of the fluorocarboxylic acid and carboxylic acid, and not more than about 25, not more than about 20, not more than about 15, not more than about 10, or not more than about 5 weight percent of the other of the fluorocarboxylic acid or carboxylic acid.

One example of a separation of acetic and trifluoroacetic acid performed in the system 100 shown in FIG. 3 is illustrated in the phase diagram provided in FIG. 2. As shown in FIG. 2, when a feed stream having a composition at point F is subjected to distillation, the resulting overhead vapor and bottoms liquid streams have respective compositions at points D and B. As shown in FIG. 2, the overhead vapor stream composition D is nearly pure trifluoroacetic acid, while the composition of the bottoms liquid stream B approaches the azeotropic composition. However, because the composition of the bottoms liquid stream is approaching the azeotrope, this stream cannot be further separated by distillation.

However, as shown in FIG. 2, the mass fraction of acetic acid in the bottoms liquid stream is higher than the mass fraction of acetic acid at the eutectic point and, therefore, the bottoms liquid stream may be further purified by crystallization. Accordingly, as shown by line 180, the bottoms liquid stream may be subjected to crystallization to provide a solid phase having a composition C and a liquor stream having a composition R. As shown in FIG. 2, the solid phase composition C is nearly pure acetic acid, while the liquor phase composition R approaches the composition of the system at its eutectic point. At this point, the liquid phase composition cannot be further purified by crystallization. However, as shown in FIG. 2, the mass fraction of acetic acid in the composition R of the liquor stream is lower than the mass fraction of acetic acid at the azeotrope and, as a result, this stream may again be subjected to distillation, as shown by line 182, for further recovery of the trifluoroacetic and acetic acid.

Referring now to FIG. 4, a schematic depiction of another separation system 200 configured according to embodiments of the present invention is provided. In the embodiment depicted in FIG. 4, separation system 200 includes a first separation zone 20 and a second separation zone 30. As shown in the embodiment depicted in FIG. 4, first separation zone 20 is a crystallization zone including at least one crystallizer 250 and second separation zone 30 is a distillation zone including at least one vapor-liquid separator 240. The feed stream in line 110 introduced into first separation zone 20 comprises a fluorocarboxylic acid and carboxylic acid that exhibit an azeotrope and a eutectic point. In some embodiments, the feed stream in line 110 may comprise an amount of the carboxylic acid in an amount higher than or lower than the amount of carboxylic acid at the eutectic point.

As shown in FIG. 4, the feed stream in line 110 may be introduced into crystallizer 250, wherein the stream may be cooled and at least partially crystallized. Crystallizer 250 may be configured in any suitable manner and can, in some embodiments, be configured as described previously with respect to FIG. 3. In the embodiment depicted in FIG. 4, the stream in line 110 may be introduced into the first stage 252a of crystallizer 250, wherein it may be cooled and at least partially frozen to form a predominantly liquid phase and a predominantly solid phase. The predominantly liquid phase may be withdrawn from first stage 252a via line 213a and the predominantly solid phase may be withdrawn from first stage 252a via line 215. The stream in line 215 may be introduced into the second stage 252b of crystallizer 250, wherein it may be further crystallized to form a second predominantly solid phase in line 112 and a second predominantly liquid phase in line 213b. The second predominantly solid phase in line 112 may be a pure component stream comprising at least about 70, at least about 75, at least about 80, at least about 85, at least about 90, or at least about 95 weight percent of the carboxylic or fluorocarboxylic acid.

As shown in FIG. 4, the second predominantly liquid phase withdrawn from the second stage 252b of crystallizer 250 in line 213b may be combined with the predominantly liquid phase withdrawn from the first stage 252a of crystallizer 250 to form a mixed component stream in line 114. In some embodiments, the mixed component stream in line 114 can have a composition of fluorocarboxylic acid and carboxylic acid approaching the eutectic point. The mixed component stream in line 114 may optionally be heated in a heat exchanger (not shown) before being introduced into a vapor-liquid separator 240, wherein it may be separated into a predominantly vapor overhead stream in line 116 and a predominantly liquid bottoms stream in line 118. Vapor-liquid separator 240 may be configured in any suitable manner and can, in some embodiments, be configured as described previously with respect to FIG. 3. The overhead vapor stream in line 116 shown in FIG. 4 may be a pure component stream including at least about 75, at least about 80, at least about 85, at least about 90, or at least about 95 weight percent of one of the fluorocarboxylic acid and carboxylic acid, while the bottoms liquid stream in line 118 may comprise a mixture of fluorocarboxylic acid and carboxylic acid having a composition approaching the azeotrope.

As shown in FIG. 4, the mixed component stream in line 118 withdrawn from vapor-liquid separator 240 in second separation zone 30 may be optionally cooled in a heat exchanger (not shown) before being returned to first separation zone 20 for further processing therein. Although shown in FIG. 4 as being combined with the feed stream in line 110 prior to being introduced into crystallizer 250 in first separation zone 20, the stream in line 118 may also be separately introduced into one or more stages of crystallizer 250, wherein it may be further separated as described above.

Separation systems and methods as described herein may be used in a variety of processes that require the high purity separation of fluorocarboxylic acid and carboxylic acid. For example, in some embodiments, systems and methods as described herein may be suitable for separating fluorocarboxylic acids and carboxylic acids from various types of process effluent streams, including, for example, wastewater or other streams intended for disposal. In other embodiments, the systems and methods described herein may be useful for recovering high purity streams of the fluorocarboxylic acid and/or carboxylic acid that may be used or reused as a catalyst, solvent, or other type of preparative agent in a chemical process.

One example of such a chemical process requiring high purity fluorocarboxylic acid and carboxylic acid separation is the production of cellulose esters such as cellulose acetate, cellulose propionate, cellulose butyrates, cellulose hexanoates, and cellulose benzoates, using trifluoroacetic anhydride. Such processes may include reacting cellulose with trifluoroacetic anhydride and a corresponding carboxylic acid or anhydride to provide the desired ester. In some embodiments, the resulting byproduct stream from the esterification reaction, which includes trifluoroacetic acid and a carboxylic acid, such as acetic acid, may be subjected to a separation process as described herein to provide high purity streams of carboxylic acid and trifluoroacetic acid. The trifluoroacetic acid may subsequently be converted to trifluoroacetic anhydride and returned to the esterification reaction, optionally with at least a portion of the recovered carboxylic acid. Additional details regarding processes and systems for producing cellulose acetate are described in PCT Application Publication No. 91/014709, the entire disclosure of which is incorporate herein by reference to the extent not inconsistent with the present disclosure.

The following examples are given to illustrate the invention and to enable any person skilled in the art to make and use the invention. It should be understood, however, that the invention is not to be limited to the specific conditions or details described in these examples. The patentable scope of the invention is defined by the claims, and may include other examples that occur to those skilled in the art.

### EXAMPLES

### Example 1

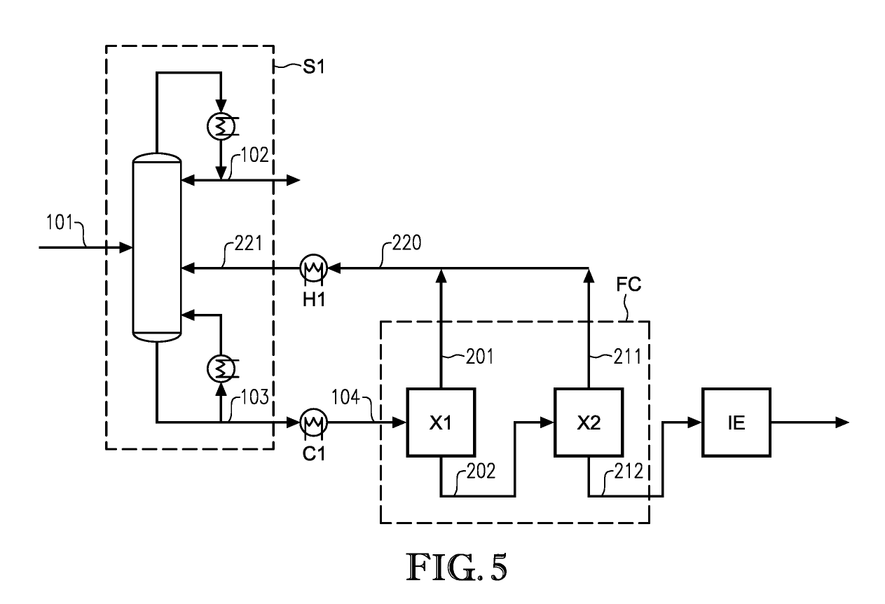

A computer simulation of the separation system shown in FIG. 5 was performed using ASPEN® Plus process modeling software (available from Aspen Technology, Inc.). The system shown in FIG. 5 is configured for separating a feed stream including trifluoroacetic acid and acetic acid to provide product streams including substantially purified acetic and trifluoroacetic acid. As shown in FIG. 5, the separation system simulated in this Example includes a first separation zone including a distillation zone S1 and a second separation zone including a fractional crystallization process FC.

In the simulation, the feed stream in line 101 had a mass fraction of acetic acid of 0.50 and a flow rate of 100 kg/hour. The distillation zone S1 shown in FIG. 5 was modeled as a continuous distillation column having 20 theoretical stages and including a reboiler and condenser for a total of 22 theoretical stages. In the simulation, distillation zone S1 had a condenser pressure at atmospheric pressure (101.325 Pa) and was modeled with a 500 Pa pressure drop for each theoretical stage. The mass reflux ratio was simulated at 5.6.

In the simulation, a fluid stream in line 221 was also introduced into distillation zone S1 as shown in FIG. 5. As shown in Table 1, the stream in line 221 had a mass fraction of acetic acid of about 0.75, which corresponds to a weight ratio of acetic acid to trifluoroacetic acid of 75:25, and a mass flow rate of 258 kg/h. In the simulation, the stream in line 221 was a mixed component stream withdrawn from the downstream crystallization zone FC and returned to distillation zone S1 for further separation.

The overhead stream withdrawn from the distillation zone S1 in line 102 had a trifluoroacetic acid purity of at least 99.99 weight percent and a mass flow rate of 50 kg/h. The total recovery of trifluoroacetic acid in the stream in line 102 was 99 weight percent, based on the total amount of trifluoroacetic acid introduced into distillation zone S1 in line 101. The bottoms stream withdrawn from distillation zone S1 in line 103 was simulated with a mass fraction of acetic acid of 0.70 (a weight ratio of acetic acid to trifluoroacetic acid of 7:3) and a mass flow rate of 162 kg/h. The temperature of the bottoms stream in line 103 was 120.2° C. Although theoretically possible to achieve a higher purity acetic acid stream limited by the azeotropic composition, the composition of the bottoms steam in line 103 has been optimized to minimize the total number of stages and energy usage, while still achieving desired purity for the product streams.

In the simulation, as shown in FIG. 5, the bottoms stream in line 103 was cooled in cooler C1 before being fed into the crystallization zone FC in the second separation zone. Crystallization zone FC includes two stages, X1 and X2, each of which was modeled to have a separation efficiency of 99 percent. The cooling temperature for each of stages X1 and X2 was modeled at −20° C. In the simulation, the solid and liquid phase streams were withdrawn from the first crystallization stage X1 via lines 202 and 201, respectively. The solid phase withdrawn from stage X1 had an acetic acid mass fraction of about 0.99 and was introduced into the second crystallization stage X2, as shown in FIG. 5. The solid phase product withdrawn from the second crystallization stage X2 had a mass fraction of acetic acid of 0.9999, which corresponds to a recovery greater than 99 percent of the total acetic acid introduced into the system in line 101. The solid phase in line 212 can optionally be melted to recover purified acetic acid and may optionally be sent to an ion exchange process (IE) shown in FIG. 5 to recover and remove any residual trifluoroacetic acid.

In the simulation, the liquid phase streams in lines 201 and 211 withdrawn from crystallization stages X1 and X2 were combined into a single stream in line 220 and returned to distillation zone S1. Prior to entering distillation zone S1, the stream in line 220 was heated in a heater H1. The results of the simulation are provided in Table 1, below.

TABLE 1
Summary of Key Parameters from Process Simulation

| | Stream | | | | | | | | | |
|---|---|---|---|---|---|---|---|---|---|---|
| | 101 | 102 | 103 | 104 | 201 | 202 | 211 | 212 | 220 | 221 |
| Mass Flow Rate | | | | | | | | | | |
| (kg/h) | | | | | | | | | | |
| Acetic Acid | 50.0 | 50.0 | 50.2 | 50.2 | 49.7 | 0.5 | 0.5 | 0.0 | 50.2 | 50.2 |
| Trifluoroacetic Acid | 50.0 | 0.0 | 117.1 | 117.1 | 65.6 | 51.4 | 1.4 | 50.0 | 67.1 | 67.1 |
| Total | 100.0 | 50.0 | 167.2 | 167.2 | 115.3 | 51.9 | 1.9 | 50.0 | 117.2 | 117.2 |
| Temperature (° C.) | 40.0 | 71.8 | 120.2 | 10.0 | −20.0 | −20.0 | −20.0 | −20.0 | −20.0 | 137.6 |

### Definitions

As used herein, the terms “comprising,” “comprises,” and “comprise” are open-ended transition terms used to transition from a subject recited before the term to one or more elements recited after the term, where the element or elements listed after the transition term are not necessarily the only elements that make up the subject.

As used herein, the terms “including,” “includes,” and “include” have the same open-ended meaning as “comprising,” “comprises,” and “comprise.”

As used herein, the terms “having,” “has,” and “have” have the same open-ended meaning as “comprising,” “comprises,” and “comprise.”

As used herein, the terms “containing,” “contains,” and “contain” have the same open-ended meaning as “comprising,” “comprises,” and “comprise.”

As used herein, the terms “a,” “an,” “the,” and “said” mean one or more.

As used herein, the term “and/or,” when used in a list of two or more items, means that any one of the listed items can be employed by itself or any combination of two or more of the listed items can be employed. For example, if a composition is described as containing components A, B, and/or C, the composition can contain A alone; B alone; C alone; A and B in combination; A and C in combination; B and C in combination; or A, B, and C in combination.

The preferred forms of the invention described above are to be used as illustration only, and should not be used in a limiting sense to interpret the scope of the present invention. Obvious modifications to the exemplary embodiments, set forth above, could be readily made by those skilled in the art without departing from the spirit of the present invention.

The inventors hereby state their intent to rely on the Equivalents to determine and assess the reasonably fair scope of the present invention as pertains to any apparatus not materially departing from but outside the literal scope of the invention as set forth in the following claims.

1. A method for separating a fluorocarboxylic acid and a carboxylic acid, said method comprising:
(a) separating a feed stream comprising said fluorocarboxylic acid and said carboxylic acid in a first separation zone to provide at least a first pure component stream and a first mixed component stream, wherein said first separation zone comprises one of a distillation zone and a crystallization zone;
(b) separating at least a portion of said first mixed component stream in a second separation zone to provide at least a second pure component stream and a second mixed component stream, wherein said second separation zone comprises the other of a distillation zone and a crystallization zone; and
(c) recycling at least a portion of said second mixed component stream from said second separation zone to said first separation zone.

2. The method of claim 1, wherein said first separation zone comprises said distillation zone and said second separation zone comprises said crystallization zone, wherein said separating of step (a) comprises separating said feed stream into an overhead stream and a bottoms stream, wherein said overhead stream comprises said first pure component stream and said bottoms stream comprises said first mixed component stream.

3. The method of claim 2, wherein said fluorocarboxylic acid and said carboxylic acid form an azeotrope or a pinch point, wherein the mass fraction of said carboxylic acid in said feed stream is lower than the mass fraction of said carboxylic acid at said azeotrope or said pinch point, and wherein said first pure component stream is enriched in said fluorocarboxylic acid.

4. The method of claim 3, wherein said fluorocarboxylic acid and said carboxylic acid also form a eutectic point.

5. The method of claim 1, wherein said first separation zone comprises said crystallization zone and said second separation zone comprises said distillation zone, wherein said separating of step (a) comprises separating said feed stream into a predominantly solid phase and a predominantly liquid phase, and wherein said first pure component stream comprises said predominantly solid phase and said first mixed component stream comprises said predominantly liquid phase.

6. The method of claim 5, wherein said fluorocarboxylic acid and said carboxylic acid form a eutectic point, wherein the mass fraction of said carboxylic acid in said feed stream is higher than the mass fraction of said carboxylic acid at said eutectic point, and wherein said first pure component stream is enriched in said carboxylic acid.

7. The method of claim 1, wherein said fluorocarboxylic acid is selected from the group consisting of trifluoroacetic acid and triflic acid.

8. The method of claim 1, wherein said fluorocarboxylic acid comprises trifluoroacetic acid and said carboxylic acid comprises acetic acid, wherein each of said trifluoroacetic acid and said acetic acid are present in said feed stream in an amount of at least 0.5 weight percent, based on the total weight of said feed stream.

9. The method of claim 8, wherein said feed stream further comprises one or more components in addition to said trifluoroacetic acid and said acetic acid.

10. The method of claim 1, further comprising, prior to step (a), synthesizing a cellulose ester, wherein said feed stream separated in said first separation zone in step (a) comprises said byproduct stream.

11. A method for separating a fluorocarboxylic acid and a carboxylic acid, said method comprising:
(a) introducing a first fluid stream comprising at least one fluorocarboxylic acid and at least one carboxylic acid into a distillation zone;
(b) separating said first fluid stream in said distillation zone to provide an overhead stream and a bottoms stream;
(c) introducing at least a portion of said bottoms stream into a crystallization zone;
(d) separating at least a portion of said bottoms stream in said crystallization zone to form a predominantly solid phase and a predominantly liquid phase; and
(e) introducing at least a portion of said predominantly liquid phase into said distillation zone.

12. The method of claim 11, wherein said fluorocarboxylic acid is selected from the group consisting of trifluoroacetic acid and triflic acid and wherein said carboxylic acid comprises acetic acid.

13. The method of claim 11, wherein said first fluid stream comprises said at least a portion of said predominantly liquid phase introduced into said distillation zone.

14. The method of claim 11, wherein said at least a portion of said predominantly liquid phase is introduced into said distillation zone in a second fluid stream.

15. The method of claim 11, wherein said fluorocarboxylic acid comprises trifluoroacetic acid and said carboxylic acid comprises acetic acid, wherein said trifluoroacetic acid and said acetic acid form a eutectic point, wherein the mass fraction of said acetic acid in said bottoms stream is higher than the mass fraction of said acetic acid at said eutectic point, and wherein said predominantly solid phase is enriched in said acetic acid.

16. The method of claim 11, wherein said fluorocarboxylic acid comprises trifluoroacetic acid and said carboxylic acid comprises acetic acid, wherein said trifluoroacetic acid and said acetic acid form an azeotrope, wherein the mass fraction of said acetic acid in said first fluid stream is lower than the mass fraction of said acetic acid at said azeotrope, and wherein said overhead stream is enriched in said trifluoroacetic acid.

17. The method of claim 11, wherein said crystallization zone comprises two or more crystallization steps performed in series or in parallel.

18. A system for separating a fluorocarboxylic acid and a carboxylic acid, said system comprising:
a distillation zone for receiving a first fluid stream comprising at least one fluorocarboxylic acid and at least one carboxylic acid, wherein said distillation zone is configured to separate said first fluid stream into a predominantly vapor first pure component overhead stream and a predominantly liquid first mixed component bottoms stream;
a crystallization zone for receiving said predominantly liquid first mixed component bottoms stream, wherein said crystallization zone is configured to separate said predominantly liquid first mixed component bottoms stream into a predominantly solid pure component stream and a predominantly liquid mixed component stream; and
a recycle conduit for passing at least a portion of said predominantly liquid mixed component stream from said crystallization zone to said distillation zone.

19. The system of claim 18, wherein said crystallization zone is a multi-stage crystallization zone.

20. The system of claim 18, further comprising a feed conduit for introducing a feed stream comprising said fluorocarboxylic acid and said carboxylic acid into said system, wherein said feed stream comprises said first fluid stream and said feed conduit is configured to introduce said first fluid stream into said distillation zone.
